(19) United States
(12) Patent Application Publication
(10) Pub. No.: US 2006/0161011 A1
(43) Pub. Date: Jul. 20, 2006

(54) Process for the ruthenium-catalysed epoxidation of olefins by means of hydrogen peroxide

(76) Inventors: Wolfgang Magerlein, Koln (DE); Matthias Beller, Nienhagen (DE); Man-Kin Tse, Rostock (DE); Santosh Bhor, Rostock (DE); Markus Klawonn, Rostock (DE); Gopinathan Anilkumar, Rostock (DE)

Correspondence Address: LANXESS CORPORATION, 111 RIDC PARK WEST DRIVE, PITTSBURGH, PA, 15275-1112, US

(21) Appl. No.: 11/330,023

(22) Filed: Jan. 11, 2006

(30) Foreign Application Priority Data
Jan. 20, 2005 (DE) 10 2005 002 821.7

(51) Int. Cl. C07D 301/12; C07F 15/00

(52) U.S. Cl. 549/531; 546/2

(57) ABSTRACT

The present invention relates to a process for the epoxidation of olefins using catalysts based on ruthenium complexes in the presence of hydrogen peroxide.

The present invention relates to a process for the epoxidation of olefins using catalysts based on ruthemium complexes in the presence of hydrogen peroxide.

Olefins are readily available and inexpensive raw materials for industrial applications. A particularly important reaction for organic syntheses is the oxidation of olefins to epoxides which are versatile intermediates in the synthesis of active compounds and fine chemicals (cosmetics industry, polymer industry, etc.)

Apart from molecular oxygen, hydrogen peroxide represents an ecologically sustainable “green” oxidant which is also inexpensive and widely available. In epoxidation reactions, atom efficiencies of up to 47% are acheived using hydrogen peroxide and only water is formed as by-product. Compared to reactions using pure oxygen (in particular pressure reactions using oxygen), hydrogen peroxyde has the advantage of a far lower safety risk.

Epoxides can traditionally be prepared from olefins by reaction with peracids which can be generated by the action of hydrogen peroxide on acids or acid derivatives. A disadvantage of this method is the restriction of the range of substrates to olefins and epoxides with are not acid-sensitive and the formation of stoichiometric amounts of salt waste. To remedy these disadvantages and to increase the selectivity of epoxidation reactions, variants which use hydrogen peroxide and are catalysed by transition metals have been developed. The most widely usable catalyst for olefin epoxidation under neutral conditions is probably the MTO system (methyltrioxorhenium) [(a) Herrman, W. A.; Fischer, R. W.; Narz, D. W. Angew. Chem. Int. Ed. 1991, 30, 1638-1641; (b) Rudolf, J.; Reddy, K. L.; Chiang, J. P.; Sharpless, K. B. J. Am. Chem. Soc. 1997, 119, 6189-6190. (c) Herrman, W. A.; Kratzer, R. M.; Ding, H.; Thiel, W. R.; Glas, H. J. Organomet. Chem. 1998, 555, 293-295.] However, from an industrial point of view, the development of a cheaper and more active and also more productive catalyst system for chemoselective olefin epoxidations using hydrogen peroxide is an important and demanding objective.

Ruthenium represents an interesting and inexpensive noble metal for epoxidation reactions. An example of a ruthenium-catalysed epoxidation in the presence of hydrogen peroxide my be found in: Stoop, R. M.; Bachmann, S.; Valentini, M.; Mezzetti, A. Organometallics 2000, 19, 4117-4126.). However, only the reaction of styrene derivatives is described here and the yields reach a maximum of 55%. A further catalytic epoxidation system based on RuCl₃ and pyridine-2,6-dicarboxylic acid, which proceeds in the presence of hydrogen peroxide, is described in Klawonn, M.; Tse, M. K.; Bhor, S.; Döbler, C.; Beller, M. J. Mol. Catal. A 2004, 218, 13-19. However, a disadvantage here is the restriction of the range of olefin substrates to olefins which are not acid-sensitive, since this reaction proceeds under acidic conditions and thus limits the tolerance of functional groups. Furthermore, a large amount of the ligand (pyridine-2,6-dicarboxylic acid) has to be added to achieve a satisfactory product yield.

There is therefore still a need to develop a general chemoselective and at the same time efficient process for the epoxidation of olefins which operates under mild and possibly pH-neutral conditions. Furthermore, the use of an oxidant which is both inexpensive and environmentally friendly is an objective to be aimed at from an industrial point of view.

It has now been found that ruthenium catalyst systems which are modified with terpyridine ligands and with 2,6-pyridine-dicarboxylic acid ligands bring about the conversion of oelfins into epoxides in the presence of hydrogen peroxide under mild, including pH-neutral conditions efficiently and with high productivities.

The process found is based on the preparation of epoxides of the formula (I), 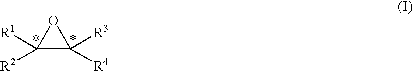
where
R¹, R², R³ and R⁴ are each, independently of one another, hydrogen, alkyl, aryl, arylalkyl, haloalkyl or a radical of one of the formulae (IIa) to (IIf)
   A-B-D-E (IIa)
   A-E (IIb)
   A-SO₂-E (IIc)
   A-B-SO₂R⁶ (IId)
   A-SO₃W (IIe)
   A-COW (IIf)
   where, in the formulae (IIa) to (IIf)
A is absent or is an alkylene or haloalkylene radical and
B is absent or is oxygen or NR5, where
R⁵ is hydrogen, arylalkyl or aryl, and
D is a carbonyl group and
E is R⁶, OR⁶, NHR⁷ or N(R⁷)₂,
   where
R⁶ is alkyl, arylalkyl or aryl and
the radicals R⁷ are each, independently of one another, alkyl, arylalkyl or aryl or the moiety N(R⁷)₂ is a cyclic amino radical having from 4 to 12 carbon atoms, and
W is OH, NH₂, or OM, where M is an alkali metal ion, half an equivalent of an alkaline earth metal ion, an ammonium ion or an organic ammonium ion,
   or two of the radicals R¹, R², R³ and R⁴ are together part of a 3- to 7-membered ring having a total of from 3 to 16 carbon atoms,
   wherein compounds of the formula (III), 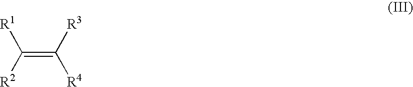
   where R¹, R², R³ and R⁴ are, in each case independently of one another, as defined above,
   are reacted with hydrogen peroxide (H₂O₂),
   with the reaction being carried out in the presence of a ruthenium complex which bears as ligands both compounds of the formula (IV) 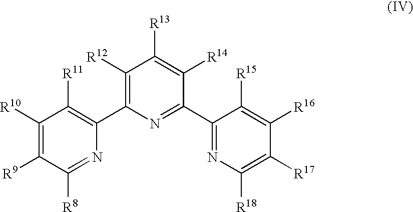
   where R⁸, R⁹, R¹⁰, R¹¹, R¹², R¹³, R¹⁴, R¹⁵, R¹⁶, R¹⁷ and R¹⁸ are each, independently of one another, hydrogen, halogen, hydroxy, hydroxycarbonyl, alkoxycarbonyl, alkoxy, alkyl, arylalkyl or aryl, or
   two of the radicals R⁸, R⁹, R¹⁰ and R¹¹ or two of the radicals R¹⁵, R¹⁶, R¹⁷ and R¹⁸ are together part of a 3- to 7-membered monocycle having a total of from 3 to 16 carbon atoms or are together part of a bicycle having a total of from 3 to 16 carbon atoms,
   and also compounds of the formula (V) 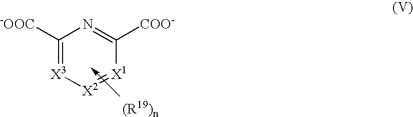
   where
X¹, X² and X³ are each, independently of one another, N, CH or CR¹⁹ and
R¹⁹ is hydrogen, halogen, hydroxy, hydroxycarbonyl, alkoxycarbonyl, alkoxy, alkoxyalkyl, arylalkyl or aryl and
n is 0, 1, 2 or 3, preferably 0 or 1 and particularly preferably 0.

The scope of the invention encompasses all definitions of radicals, parameters and explanations given above and in the following either in general terms or in preferred ranges in any combination with one another and also between the respective ranges and preferred ranges.

For the purposes of the invention, the term aryl preferably refers, unless indicated otherwise, to carbocyclic aromatic radicals having from 6 to 24 skeletal carbon atoms or heteroaromatics having from 5 to 24 skeletal carbon atoms in which no, one, two or three skeletal carbon atoms per ring, but at least one skeletal carbon atom in the total molecule, can be replaced by heteroatoms selected from the group consisting of nitrogen, sulphur and oxygen. Furthermore, the carbocyclic aroamtic radicals or heteroaromatic radicals can be substituted by up to 5 identical or different substituents selected from the group consisting of hydroxy, halogen, nitro, cyano, free or protected formyl, C₁-C₁₂-alkyl, C₁-C₁₂-haloalkyl, C₅-C₁₄-aryl, C₆-C₁₅-arylalkyl, C₁-C₁₂-alkoxy, C₁-C₁₂-alkoxycarbonyl, —PO—[(C₁-C₈)-alkyl]₂, —PO—[(C₅-C₁₄)-aryl]₂, —PO—[(C₁-C₈)-alkyl)(C₅-C₁₄)-aryl)], tri(C₁-C₈-alkyl)siloxyl and radicals of the formulae (IIa) to (IIf) per ring. The same applies to the aryl part of an arylalkyl radical.

For example, aryl is particularly preferably phenyl, naphthyl or anthracenyl which may be substituted by one, two or three radicals selected independently from the group consisting of C₁-C₆-alkyl, C₁-C₆-haloalkyl, C₅-C₁₄-aryl, C₁-C₆-alkoxy, C₁-C₆-alkoxycarbonyl, halogen, hydroxy, nitro and cyano.

For the purposes of the invention, the terms alkyl and alkylene and alkoxy preferably refer, unless indicated otherwise and in each case independently, to a substituted or unsubstituted straight-chain, cyclic, branched or unbranched alkyl or alkylene or alkoxy radical. The same applies to the alkylene part of an arylalkyl radical. Possible substituents for the alkyl or alkylene or alkoxy radicals are, for example, C₁-C₆-alkyl, C₁-C₆-haloalkyl, C₅-C₁₄-aryl, C₆-C₁₅-arylalkyl, C₁-C₆-alkoxy, C₁-C₆-aryloxy, C₁-C₆-alkoxycarbonyl, C₁-C₆-acyloxy, halogen, hydroxy, nitro, cyano or tri(C₁-C₈-alkyl)siloxyl.

For example, alkyl is particularly preferably methyl, ethyl, n-propyl, isopropyl, n-butyl, tert-butyl, n-pentyl, cyclohexyl or n-hexyl, n-heptyl, n-octyl, isooctyl, n-decyl or n-dodecyl.

Alkylene is preferably, for example, methylene, 1,1-ethylene, 1,2-ethylene, 1,1-propylene, 1,2-propylene, 1,3-propylene, 1,1-butylene, 1,2-butylene, 2,3-butylene or 1,4-butylene, 1,5-pentylene, 1,6-hexylene, 1,1-cyclohexylene, 1,4-cyclohexylene, 1,2-cyclohexylene or 1,8-octylene.

Alkoxy is preferably, for example, methoxy, ethoxy, isopropoxy, n-propoxy, n-butoxy, tert-butoxy or cyclohexyloxy.

Cyclic alkyl radicals can be either 3- to 7-membered homocycles or heterocycles having a total of from 3 to 17 carbon atoms, the latter preferably having 1, 2 or 3 heteroatoms. Homocyclic alkyl radicals are, for example, substituted or unsubstituted cyclopentyl or cyclohexyl, and examples of heterocyclic alkyl radicals are dioxolane or phthalimide radicals.

In tri(C₁-C₈-alkyl)siloxyl substituents, the C₁-C₈-alkyl radicals can be identical or different. An example of such a substituent is tert-butyldimethylsiloxy.

For the purposes of the invention, the term arylalkyl preferably refers, unless indicated otherwise and in each case independently, to a straight-chain, cyclic, branched or unbranched alkyl radical which may be monosubstituted or polysubstituted, particularly preferably monosubstituted, by aryl radicals as defined above.

For the purposes of the invention, the terms haloalkyl and haloalkylene preferably refer, unless indicated otherwise and in each case independently, to a straight-chain, cyclic, branched or unbranched alkyl radical which may be monosubstituted, polysubstituted or fully substituted by halogen atoms selected independently from the group consisting of fluorine, chlorine, bromine and iodine.

For example, C₁-C₈-haloalkyl is particularly preferably trifluoromethyl, trichloromethyl, 2,2,2-trifluoroethyl, pentafluoroethyl or nonafluorobutyl.

Halogen can be fluorine, chlorine, bromine or iodine, preferably fluorine or chlorine.

Protected formyl is a formyl radical which has been protected by conversion into an aminal, acetal or mixed aminal-acetal, with the aminals, acetals and mixed aminal-acetals being able to be acyclic or cyclic.

The carbon atoms denoted by * in the general formula (I) can, depending on the meaning of R¹ to R⁴, be, independently of one another, asymmetric carbon atoms which can have, independently of one another, the (R) or (S) configuration. For the purposes of the invention, it is possible for both, one of the two or none of the two carbon atoms denoted by * in the general formula (I) to be asymmetric.

Preferred compounds of the formulae (I), (IV) and (V) are defined below.

In the formula (I), preference is given to R¹, R², R³ and R⁴ each being, independently of one another, hydrogen, substituted or unsubstituted C₁-C₈-alkyl, C₅-C₁₄-aryl, C₆-C₁₅-arylalkyl, C₁-C₈-haloalkyl or two of the radicals R¹, R², R³ and R⁴ are together part of a 3- to 7-membered ring having a total of from 3 to 16 carbon atoms.

Particular preference is given to compounds of the formula (I) in which at least one radical R¹, R², R³ and R⁴ is substituted or unsubstituted C₅-C₁₄-aryl or two of the radicals R¹, R², R³ and R⁴ are together part of a 3- to 7-membered ring having a total of from 3 to 16 carbon atoms.

In the formula (IV), preference is given to R⁸, R⁹, R¹¹, R¹², R¹⁴, R¹⁵, R¹⁷ and R¹⁸ each being hydrogen and at the same time R¹⁰, R¹³ and R¹⁶ each being tert-butyl, R⁸, R⁹, R¹⁰, R¹¹, R¹², R¹³, R¹⁴, R¹⁵, R¹⁶, R¹⁷ and R¹⁸ each being hydrogen or two of the radicals R⁸, R⁹, R¹⁰ and R¹¹ and two of the radicals R¹⁵, R¹⁶, R¹⁷ and R¹⁸ together being part of a 3- to 7-membered monocycle having a total of from 3 to 16 carbon atoms or together being part of a bicycle having a total of from 3 to 16 carbon atoms and the remaining radicals R⁸, R⁹, R¹⁰, R¹¹, R¹², R¹³, R¹⁴, R¹⁵, R¹⁶, R¹⁷ and R¹⁸ are each hydrogen. In a preferred embodiment, R⁸, R⁹, R¹⁰, R¹¹, R¹², R¹³, R¹⁴, R¹⁵, R¹⁶, R¹⁷ and R¹⁸ are each hydrogen.

In compounds of the formula (V), preference is given to at least two, particularly preferably three, of the radicals X¹, X², X³ being CH or CR¹⁹ and very particularly preferably CH. n is preferably 0 or 1, particularly preferably 0 or 1 with a substituent in the 4 position and very particularly preferably 0.

Preferred ruthenium complexes are complexes of the formula (VI)
[Ru(IV)(V)] (VI)
where (IV) represents a compound of the formula (IV) and (V) represents a compound of the formula (V). Such complexes can be prepared in a manner known per se using methods analogous to those described in the references cited at the outset (Nishiyama, H.; Shimada, T.; Itoh, H.; Sugiyama, H.; Motoyama, Y. Chem. Commun. 1997, 1863-1864).

In a preferred embodiment, the process of the invention is carried out in the presence of an organic solvent such as, in particular, secondary or tertiary alcohols, aprotic polar solvents, ketones, chlorinated hydrocarbons and aromatic hydrocarbons. Aprotic polar solvents are solvents which have a dielectric constant at 25° C. of 5 or more and a pKₐ based on an aqueous reference scale at 25° C. of 20 or more. Particular preference is given to secondary and tertiary alcohols such as, in particular, t-amyl alcohol and t-butyl alcohol in the process of the invention.

The reaction is, for example, carried out by placing the compounds of the formula (III) and the ruthenium complex together with an organic solvent in a reaction vessel and adding the oxidant which may, if desired, be dissolved in a suitable organic solvent. In a preferred embodiment, a solution of the oxidant is introduced into the reaction mixture over a period of from 10 minutes to 24 hours.

Any additional subsequent stirring time can be, for example, up to 24 hours, preferably up to 5 hours and particularly preferably up to 1 hour.

The reaction can be carried out at temperatures of from −20° C. to 150° C., preferably from 0 to 80° C., particularly preferably from 0° C. to 40° C. and very particularly preferably from 15° C. to 30° C.

The pressure during the reaction is not critical and can be, for example, from 0.5 to 100 bar, preferably from 0.8 to 10 bar. Particular preference is given to ambient pressure.

The oxidant hydrogen peroxide is preferably used in an amount of from 1 to 10 molar equivalents based on compounds of the formula (III), particularly preferably from 1 to 5 molar equivalents and very particularly preferably from 1 to 3 molar equivalents. The oxidants may advantageously be used as a solution in a solvent, particularly preferably as a solution in water and, if appropriate, additionally at least one of the above-described organic solvents.

For the purposes of the invention, the ruthenium complex can either be used as an isolated complex or can be generated in situ in the reaction mixture. In the latter case, a suitable ruthenium precursor, e.g. [Ru(p-cymene)Cl₂]₂, and the two ligands of the formulae (IV) and (V) are combined in the reaction mixture.

The isolated complexes are preferably likewise prepared by combining a suitable ruthenium precursor, e.g. [Ru(p-cymene)Cl₂]₂, and the two ligands of the formulae (IV) and (V) by, for example, firstly placing the ruthenium precursor together with the ligand of the formula (IV) and a suitable solvent in an inert gas atmosphere in a reaction vessel and adding a solution of the ligand of the formula (V), preferably in the form of its disodium salt, subsequently heating the reaction mixture and isolating the ruthenium complex, for example by crystallization, filtration and recrystallization. Particularly when using isolated complexes as catalysts, the process of the invention can be carried out under pH-neutral conditions, which opens up a wider range of applications for the compounds of the formula (III). The use of isolated complexes of the formula (IV) is therefore preferred.

Suitable ruthenium precursors are, for example, ruthenium compounds such as Ru(III) chloride or, for example, Ru(II) or Ru(III) complexes having at least one ligand from the group consisting of phosphanes, e.g. triarylphosphanes, trialkylphosphanes or bis(diarylphosphino)alkanes, amines such as triarylamines, trialkylamines, cycloaliphatic or cycloaromatic or heteroaromatic amines or unsaturated cyclic hydrocarbons such as p-cymene, norbornadiene or cyclooctadiene. An example of a suitable ruthenium precursor is [Ru(p-cymene)Cl₂]₂.

The reaction can be carried out under pH-neutral conditions, and the addition of acids or bases may also be advantageous. The reaction is preferably carried out under pH-neutral conditions, which for the present purposes means pH values of from 5 to 9, measured at 20° C.

For the purposes of the invention, the amount of ruthenium complex used or of ruthenium precursor used is, for example, in the range from 0.001 to 20 mol %, preferably from 0.01 to 1 mol % and particularly preferably from 0.1 to 1 mol %.

Compounds of the formula (I) can be obtained in very good yields under mild conditions by the route provided by the invention. The work-up can be carried out in a manner known per se, e.g. by quenching with water, extraction with a suitable organic solvent and distillation or recrystallization of the epoxide.

The process of the invention can be carried out either stereoselectively or nonstereoselectively. The process of the invention is preferably carried out nonstereoselectively. For the purposes of the invention, carrying out the process of the invention nonstereoselectively includes carrying it out racemically. However, carrying out the process of the invention stereoselectively can also be preferred.

Preferred catalysts for such a stereoselective (enantioselective) epoxidation are catalysts of the formula (VI) in which two of the radicals R⁸, R⁹, R¹⁰ and R¹¹ in the compounds of the formula (IV) or at least two of the radicals R¹⁵, R¹⁶, R¹⁷ and R¹⁸ are together part of a 3- to 7-membered bicycle having a total of from 3 to 16 carbon atoms, with this bicycle particularly preferably being derived from a terpene.

Examples of such catalysts of the formula (VI) are the compounds of the formulae (VI-2) to (VI-5), 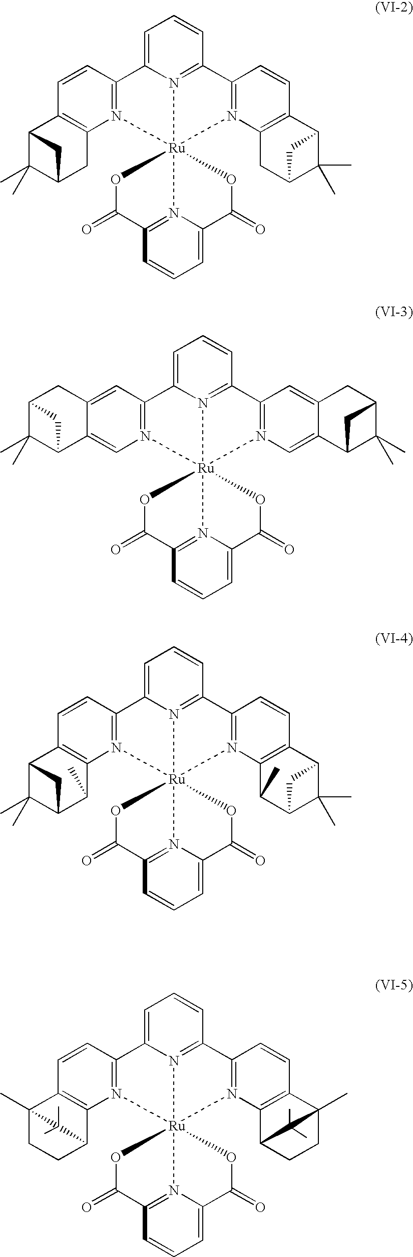
which have hitherto not been described in the literature and are therefore likewise a subject matter of the present invention.

When the process of the invention is carried out enantioselectively, one of the two enantiomers of the general formula (I) is obtained in an enantiomeric excess, hereinafter also referred to as ee, compared to the other enantiomer. This enantiomeric excess is preferably from 2 to 100% ee, particularly preferably from 50% to 100%. A definition of the ee value is given in the examples in the present patent application. If both the carbon atoms denoted by * in the general formula (I) are asymmetric, the two enantiomers can also be a diastereomeric pair.

The compounds of the formula (I) which can be prepared according to the invention are particularly suitable for producing medicaments, agrochemicals, polymers or intermediates thereof.

In the process of the invention, the epoxidation of olefins proceeds with high chemoselectivity under very mild conditions and gives very good product yields. Particular mention may be made of the very small amounts of ruthenium and of ligands required. At the same time, the ability to use the inexpensive oxidant hydrogen peroxide is a particular advantage and a further advantage is the ability to react even acid-sensitive compounds of the formula (III) and/or prepare acid-sensitive compounds of the formula (I) under pH-neutral conditions.

### EXAMPLES

General Method:

In a typical experiment, the ruthenium complex [Ru(2,2′:6′,2″-terpyridine)(pyridine-2,6-dicarboxylate)] (VI-1) (0.0025 mmol) is stirred in tert-amyl alcohol (9 ml) at room temperature and the olefin of the formula (III) (0.5 mmol) is added. A solution of 30% strength hydrogen peroxide (1.5 mmol) in t-amyl alcohol (0.83 ml) is metered into this mixture over a period of 12 hours. The reaction is then quenched by addition of water (10 ml) and Na₂SO₃ (0.5 g) and the mixture is extracted with ethyl acetate (20 ml). After the organic phase has been dried, aliquots are analysed by means of gas chromatography. To isolate the epoxides, the solvent is removed by distillation and the product is, if appropriate, purified by column chromatography.

### Examples 1-25

Table 1 summarizes the examples of the oxidation of olefins of the formula (III) in accordance with the general method in the presence of the ruthenium complex [Ru(2,2′:6′,2″-terpyridine)(pyridine-2,6-dicarboxylate)] (VI-1):

| | | | (VI-1) |
|---|---|---|---|
| 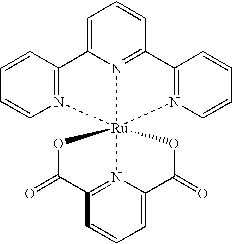 | | | |
| | Olefin of the | Conversion | Yield |
| Ex. | formula (III) | (%) | (%) |
| 1 | 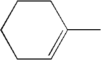 | 100 | 84 |
| 2 | 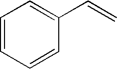 | 100 | 71 |
| 3 | 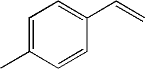 | 100 | 83 |
| 4 | 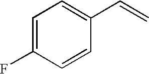 | 100 | 86 |
| 5 | 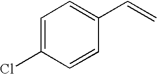 | 100 | >99 |
| 6 | 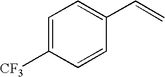 | 90 | 89 |
| 7 | 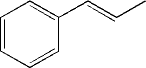 | 100 | >99 |
| 8 | 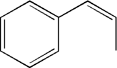 | 100 | >99 |
| 9 | 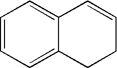 | 100 | 80 |
| 10 | 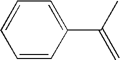 | 100 | 88 |
| 11 | 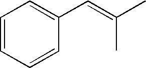 | 100 | >99 |
| 12 | 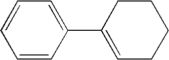 | 100 | 91 |
| 13 | 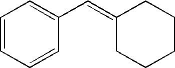 | 100 | 86 |
| 14 | 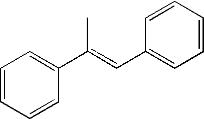 | 100 | 95 |
| 15 | 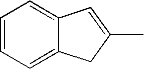 | 100 | 86 |
| 16 | 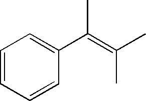 | 100 | 96 |
| 17 | 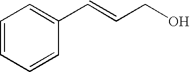 | 100 | 97 |
| 18^{(a)} | 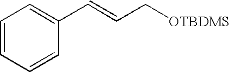 | 100 | >99 |
| 19^{(b)} | 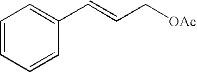 | 100 | 98 |
| 20 | 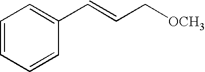 | 100 | 96 |
| 21 | 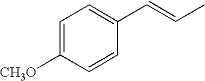 | 100 | 99 |
| 22 | 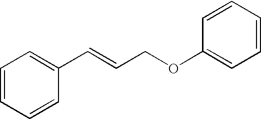 | 100 | 92 |
| 23 | 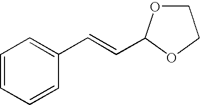 | 100 | 62 |
| 24 | 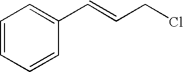 | 100 | 94 |
| 25 | 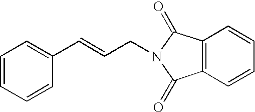 | >90 | 81 |

^{(a)}OTBDME = tert-butyldimethylsiloxyl;
^{(b)}Ac = acetyl
Tab. 1: Epoxidation of olefins of the formula (III) in accordance with the general method in the presence of the ruthenium complex [Ru(2,2′:6′,2″-terpyridine)(pyridine-2,6-dicarboxylate)](VI-1)

The analytical data for the epoxides of the formula (I) prepared in the examples are shown below.

1,2-Epoxy-1-methylcyclohexane: ¹H NMR (400.1 MHz, CDCl₃): δ 1.22 (m, 5H), 1.36-1.31 (m, 2H), 1.59 (m, 2H), 1.82-1.78 (m, 2H), 2.87 (s, 1H); ¹³C NMR (100.6 MHz, CDCl₃, ppm): δ 19.7, 20.1, 22.7, 25.0, 29.9, 57.8, 59.6; (E.I., 70 eV): m/e 112 (M⁺), 111, 97 (100), 55, 43.

Phenyloxirane: ¹H NMR (400.1 MHz, CDCl₃): δ 2.72 (dd, J=5.6, 2.6 Hz, 1H), 3.06 (dd, J=5.6, 4.2 Hz, 1H), 3.78 (dd, J=4.2, 2.6 Hz, 1H), 7.16-7.29 (m, 5H); ¹³C NMR (100.6 MHz, CDCl₃): δ 51.3, 52.5, 125.6, 128.3, 128.6, 137.7; (E.I., 70 eV): m/e 120 (M⁺, 41), 119 (65), 92 (37), 91 (100), 90 (64), 89 (79).

2-(p-Tolyl)oxirane: ¹H NMR (400.1 MHz, CD₂Cl₂): δ 2.33 (s, 3H), 2.77 (dd, J=5.5, 2.6 Hz, 1H), 3.09, (dd, J=5.5, 4.1 Hz, 1H), 3.79, (dd, J=4.1, 2.6 Hz, 1H), 7.06-7.26 (m, 5H); ¹³C NMR (100.6 MHz, CD₂Cl₂): δ 20.9, 50.9, 52.1, 125.5, 129.2, 134.8, 138.1; GC-MS: m/e 134 (M⁺).

4-Fluorophenyloxirane: ¹H NMR (400.1 MHz, CDCl₃): δ 2.67 (dd, J=5.6, 2.6 Hz, 1H), 3.04 (dd, J=5.6, 4.0 Hz, 1H), 3.75 (dd, J=4.0, 2.6 Hz, 1H), 6.91-6.96 (m, 2H), 7.12-7.17 (m, 2H), ¹³C NMR (100.6 MHz, CDCl₃): δ 51.6, 52.2, 115.9 (d, J=20 Hz), 127.6 (d, J=7 Hz), 133.7 (d, J=2 Hz), 163.1 (d, J=24 Hz); (E.I., 70 eV): m/e 138 (M⁺), 137 (M−1⁺), 122 (86), 109 (100), 96.

4-Chlorophenyloxirane: ¹H NMR (400.1 MHz, CDCl₃): δ 2.68 (dd, J=5.6, 2.6 Hz, 1H), 3.07 (dd, J=5.6, 4.0 Hz, 1H), 3.76 (dd, J=4.0, 2.6 Hz, 1H), 7.12-7.26 (m, 4H); ¹³C NMR (100.6 MHz, CDCl₃): δ 51.4, 51.9, 127.0, 128.8, 134.1, 136.3; (E.I., 70 eV): m/e 156 (M+2⁺, 9), 155 (M+1⁺, 10), 154 (M⁺, 28), 153 (M−1⁺, 23), 125 (53), 119 (74), 89 (106).

(4-Trifluoromethyl)phenyloxirane: ¹H NMR (400.1 MHz, CDCl₃): δ 2.77 (dd, J=5.6, 2.6 Hz, 1H), 3.19 (dd, J=5.6, 4.0 Hz, 1H), 3.92 (dd, J=4.0, 2.6 Hz, 1H), 7.4 (d, J=8.1 Hz, 2H), 7.6 (d, J=8.1 Hz, 2H); ¹³C NMR (100.6 MHz, CDCl₃): δ 51.4, 51.6, 125.4 (q, J=3.8 Hz), 125.9, 141.9; (E.I., 70 eV): m/e 188 (M⁺, 14), 187 (20), 159 (49), 158 (48), 119 (100), 91 (37).

Trans-2-Methyl-3-phenyloxirane: ¹H NMR (400.1 MHz, CDCl₃): δ 1.44 (d, J=5.2 Hz, 3H), 3.03 (dq, J=5.2, 2.0 Hz, 1H), 3.57 (d, J=2.0 Hz, 1H), 7.23-7.4 (m, 5H); ¹³C NMR (100.6 MHz, CDCl₃): δ 18.0, 59.2, 59.6, 125.7, 128.1, 128.5, 137.9; (E.I., 70 eV): m/e 134 (M⁺, 52), 133 (65), 105 (51), 91 (42), 90 (100), 89 (77), 77 (23).

Cis-2-Methyl-3-phenyloxirane: ¹H NMR (400.1 MHz, CDCl₃): δ 1.07 (d, J=5.4 Hz, 3H), 3.33 (dd, J=5.4, 4.3 Hz, 1H), 4.05 (d, J=4.3 Hz, 1H), 7.25-7.36 (m, 5H); ¹³C NMR (100.6 MHz, CDCl₃): δ 12.5, 55.1, 57.5, 126.5, 127.4, 128.0, 135.5; MS (E.I., 70 eV): m/e 134 (M⁺).

1,2-Dihydronaphthalene oxide: ¹H NMR (400.1 MHz, CDCl₃): δ 1.72-1.80 (m, 1H), 2.41 (dddd, J=14.5, 6.5, 2.9, 1.7 Hz, 1H), 2.55 (dd, J=15.5, 5.6 Hz, 1H), 2.76-2.85 (m, 1H), 3.72-3.74 (m, 1H), 3.85 (d, J=4.4 Hz, 1H), 7.10 (d, J=7.3 Hz, 1H), 7.19-7.23 (m, 1H), 7.25-7.29 (m, 1H), 7.40 (dd, J=7.3, 1.4 Hz, 1H); ¹³C NMR (100.6 MHz, CDCl₃): δ 21.7, 24.3, 52.6, 54.9, 126.0, 128.2, 128.3, 129.4, 132.4, 136.5; GC-MS: m/e 146 (M⁺).

2-Methyl-2-phenyloxirane: ¹H NMR (400.1 MHz, CDCl₃): δ 1.65 (s, 3H), 2.73 (d, J=5.4 Hz, 1H), 2.90 (d, J=5.4 Hz, 1H), 7.17-7.31 (m, 5H), ¹³C NMR (100.6 MHz, CDCl₃): δ 56.9, 57.2, 125.4, 127.6, 128.5, 141.3; MS (E.I., 70 eV): m/e 134 ([M]⁺, 35), 133 (87), 105 (100), 104 (41), 103 (58), 91 (23), 79 (37), 78 (54), 77 (49).

2,2-Dimethyl-3-phenyloxirane: ¹H NMR (400.1 MHz, CDCl₃): δ 1.04 (s, 3H), 1.45 (s, 3H), 3.83 (s, 1H), 7.21-7.33 (m, 5H); ¹³C NMR (100.6 MHz, CDCl₃): δ 17.9, 24.7, 61.0, 64.5, 126.3, 127.3, 128, 136.6; MS (E.I., 70 eV) m/e 148 (M⁺).

1,2-Epoxy-1-phenylcyclohexane: ¹H NMR (400.1 MHz, CDCl₃): δ 1.18-1.30 (m, 1H), 1.34-1.44 (m, 1H), 1.44-1.58 (m, 2H), 1.87-1.95 (m, 2H), 2-2.09 (m, 1H), 2.16-2.25 (m, 1H), 2.99 (m, 1H), 7.15-7.20 (m, 1H), 7.23-7.32 (m, 4H); ¹³C NMR (100.6 MHz, CDCl₃): δ 19.9, 20.2, 24.8, 29.0, 60.3, 62.1, 125.4, 127.3, 128.4, 142.6, MS (E.I., 70 eV): m/e=175 ([M+1]⁺, 10), 174 ([M]⁺, 82), 173 (100), 159 (21), 145 (40), 129 (50), 117 (47), 115 (58), 105 (68), 91 (58), 77 (43).

2-Phenyl-1-oxaspiro[2.5]octane: ¹H NMR (400.1 MHz, CDCl₃): δ 1.22-1.31 (m, 2H), 1.37-1.85 (m, 8H), 3.85 (s, 1H), 7.23-7.34 (m, 5H); ¹³C NMR (100.6 MHz, CDCl₃): δ 24.5, 25.3, 25.5, 28.4, 35.4, 64.5, 65.5, 126.3, 127.2, 127.9, 136.3; MS (E.I., 70 eV) m/e 188 (M⁺).

2-Methyl-2,3-diphenyloxirane: ¹H NMR (400.1 MHz, CDCl₃): δ 1.48 (s, 3H), 3.98 (s, 1H), 7.30-7.34 (m, 2H), 7.37-7.42 (m, 6H), 7.45-7.48 (m, 2H); ¹³C NMR (100.6 MHz, CDCl₃): δ 16.7, 63.0, 67.1, 125.1, 126.5, 127.5, 127.6, 128.2, 128.4, 135.9, 142.3; MS (E.I., 70 eV): m/e 210 (M⁺).

2-Methylindene oxide: 1H NM (400.1 MHz, CDCl₃): δ 1.69 (s, 3H), 2.90 (d, J=17.7 Hz, 1H), 3.15 (d, J=17.7 Hz, 1H), 4.04 (d, J=1.2 Hz, 1H), 7.14-7.25 (m, 3H), 7.44 (d, J=7.3 Hz, 1H); ¹³C NMR (100.6 MHz, CDCl₃): δ 18.5, 38.6, 65.0, 65.3, 124.8, 125.7, 126.0, 128.2, 141.7, 144.5; MS (E.I., 70 eV) m/e 146 (M⁺).

2,2,3-Trimethyl-3-phenyloxirane: ¹H NMR (400.1 MHz, CDCl₃): δ 0.95 (s, 3H), 1.46 (s, 3H), 1.61 (s, 3H), 7.20-7.23 (m, 1H), 7.27-7.33 (m, 4H); ¹³C NMR (100.6 MHz, CDCl₃): δ 20.7, 21.3, 21.7, 63.7, 66.5, 126.0, 126.7, 128.0, 142.2; MS (E.I., 70 eV) m/e 162 (M⁺).

Trans-2-Hydroxymethyl-3-phenyloxirane: ¹H NMR (400.1 MHz, CD₂Cl₂): [symbol] 1.84 (br s, 1H), 3.20 (d, J=4.2, 2.2 Hz, 1H), 3.74 (dd, J=12.7, 4.2 Hz, 1H), 3.88 (d, J=2.2 Hz, 1H), 4.01 (dd, J=12.7, 2.2 Hz, 1H), 7.27-7.38 (m, 5H); ¹³C NMR (100.6 MHz, CD₂Cl₂): [symbol] 55.8, 61.7, 62.8, 116.7, 126.1, 128.5, 128.8; MS (E.I., 70 eV): m/e 150 ([M]⁺, 5), 132 (19), 131 (12), 119 (19), 107 (100), 105 (33), 104 (34), 91 (67), 90 (78), 89 (58), 79 (67), 77 (41).

Trans-2-[(tert-Butyldimethylsiloxy)methyl]-3-phenyloxirane: ¹H NMR (400.1 MHz, CDCl₃): δ 0.09 (s, 3H), 0.10 (s, 3H), 0.91 (s, 9H), 3.12-3.13 (ddd, J=4.4, 2.8, 1.9 Hz, 1H), 3.79 (d, J=1.9 Hz, 1H), 3.81 (dd, J=12.0, 4.4 Hz, 1H), 3.95 (dd, J=12.0, 2.8 Hz, 1H), 7.24-7.35 (m, 5H); ¹³C NMR (100.6 MHz, CDCl₃): δ −5.3, 18.4, 25.9, 55.9, 62.7, 64.0, 125.7, 128.1, 128.4, 137.2; MS (E.I., 70 eV): m/e 249 (M⁺-CH₃).

3-Phenyloxiranylmethyl acetate: ¹H NMR (400.1 MHz, CDCl₃): δ 2.04 (s, 3H), 3.18-3.20 (m, 1H), 3.73 (d, J=2.0 Hz, 1H), 4.02 (dd, J=12.3, 6.0 Hz, 1H), 4.41 (dd, J=12.3, 3.4 Hz, 1H), 7.17-7.32 (m, 5H), ¹³C NMR (100.6 MHz, CDCl₃): δ 20.7, 56.4, 59.2, 64.2, 125.6, 128.4, 128.5, 136.1, 170.7; MS (E.I., 70 eV): m/e 192 (M⁺, 2), 150 (10), 149 (79), 133 (26), 107 (95), 105 (67), 91 (54), 90 (45), 89 (42), 79 (31), 77 (31), 43 (100).

Trans-2-Methoxymethyl-3-phenyloxirane: ¹H NMR (400.1 MHz, CDCl₃): δ 3.19 (ddd, J=5.2, 3.1, 2.1 Hz, 1H), 3.43 (s, 3H), 3.52 (dd, J=11.4, 5.2 Hz, 1H), 3.76 (dd, J=11.4, 3.1 Hz, 1H), 3.78 (d, J=2.1 Hz, 1H), 7.25-7.35 (m, 5H); ¹³C NMR (100.6 MHz, CDCl₃): δ 55.7, 59.2, 60.9, 72.1, 125.6, 128.2, 128.4, 136.8; MS (E.I., 70 eV) m/e 164 (M⁺).

Trans-2-(p-methoxyphenyl)-3-methyloxirane: ¹H NMR (400.1 MHz, CD₂Cl₂): δ 1.41 (d, J=5.2 Hz, 3H), 3.01 (qd, J=5.2, 2.0 Hz, 1H), 3.50 (d, J=2.0 Hz, 1H), 3.79 (s, 3H), 6.87 (d, J=8.9 Hz, 2H), 7.17 (d, J=8.9 Hz, 2H); ¹³C NMR (100.6 MHz, CD₂Cl₂): δ 18.0, 58.9, 59.5, 114.1, 127.2, 130.3, 160.0; MS (E.I., 70 eV): m/e=165 ([M+1]⁺, 7), 164 (M⁺, 57), 121 (47), 120 (82), 105 (31), 91 (100), 77 (55), 51 (37).

Trans-2-Phenoxymethyl-3-phenyloxirane: ¹H NMR (400.1 MHz, CDCl₃): δ 3.40 (ddd, J=5.2, 3.2, 2.0 Hz, 1H), 3.91, (d, J=2.0 Hz, 1H), 4.14 (dd, J=11.2, 5.2 Hz, 1H), 4.32 (dd, J=11.2, 3.2 Hz, 1H), 6.94-7.00 (m, 3H), 7.27-7.38 (m, 7H); ¹³C NMR (100.6 MHz, CDCl₃): δ 56.4, 60.2, 67.8, 114.7, 121.3, 125.7, 128.4, 128.5, 129.5, 136.5, 158.4; MS (E.I., 70 eV) m/e 226 (M⁺).

2-(3-Phenyloxiranyl)-[1,3]dioxolane: ¹H NMR (400.1 MHz, CDCl₃): δ 3.13 (dd, J=3.8, 2.0 Hz, 1H), 3.89 (d, J=2.0 Hz, 1H), 3.89-3.97 (m, 2H), 4.00-4.06 (m, 2H), 5.00 (d, J=3.8, 1H), 7.25-7.35 (m, 5H); ¹³C NMR (100.6 MHz, CDCl₃): δ 55.2, 61.3, 65.3, 65.5, 102.3, 125.7, 128.3, 128.4, 136.2; GC-MS: m/e 192 (M⁺).

Trans-2-Chloromethyl-3-phenyloxirane: ¹H NMR (400.1 MHz, CDCl₃): δ 3.28 (ddd, J=5.8, 4.8, 1.9 Hz, 1H), 3.66 (dd, J=11.8, 5.8 Hz, 1H), 3.72 (dd, J=11.8, 4.8, Hz, 1H), 3.82 (d, J=1.9 Hz, 1H), 7.26-7.38 (m, 5H); ¹³C NMR (100.6 MHz, CDCl₃): δ 44.3, 58.5, 60.9, 116.6, 125.6, 128.6, 135.9; GC-MS: m/e 168 (M⁺).

2-(3-Phenyloxiranylmethyl)isoindole-1,3-dione: ¹H NMR (400.1 MHz, CDCl₃): δ 3.20 (ddd, J=5.7, 4.7, 1.9 Hz, 1H), 3.82 (dd, J=14.3, 5.7 Hz, 1H), 3.83 (d, J=1.9 Hz, 1H), 4.09 (dd, J=14.3, 4.7 Hz, 1H), 7.19-7.29 (m, 5H), 7.68 (dd, J=5.5, 3.1 Hz, 2H),), 7.82 (dd, J=5.5, 3.1 Hz, 2H); ¹³C NMR (100.6 MHz, CDCl₃): δ 39.3, 58.0, 58.9, 123.5, 125.6, 128.4, 128.5, 131.9, 134.2, 136.3, 168.0; MS (E.I., 70 eV): m/e 279 (M⁺).

### Example 26

Synthesis of the Ruthenium Complex (VI-2) [Ru(tpy-β-Pinene)(Pydic)]

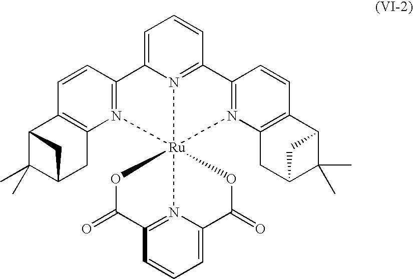

200 mg (0.47 mmol) of the ligand tpy-β-pinene and 145 mg of the ruthenium precursor complex [Ru(p-cymene)Cl₂]₂ (0.24 mmol) together with 8 ml of methanol were placed in a reaction vessel at room temperature and a solution of 100 mg of the disodium salt of pyridine-2,6-dicarboxylic acid (H₂pydic) (0.47 mmol) dissolved in 9 ml of MeOH/H₂O (2/1) was added. The reaction mixture was heated at 65° C. for 1 hour. After cooling, the product was crystallized to give [Ru(tpy-β-pinene)(pydic)] (61 mg, 19%) as a violet, crystalline solid.

R_{f}=0.20 (CH₂Cl₂/MeOH 100:5). ¹H NMR (400.1 MHz, CD₂Cl₂, ppm) δ 0.44 (s, 6H), 1.00 (d, J=9.9 Hz, 2H), 1.26 (s, 6H), 1.88-1.92 (m, 2H), 2.30-2.31 (m, 4H), 2.43-2.48 (m, 2H), 2.69-2.71 (m, 2H), 7.27 (d, J=7.9 Hz, 2H), 7.75 (t, J=8.1 Hz, 1H), 8.00 (d, J=7.9 Hz, 2H), 8.08 (t, J=7.7 Hz, 1H), 8.24 (d, J=8.1 Hz, 2H), 8.30 (d, J=7.7 Hz, 2H). ¹³C NMR (100.6 MHz, CD₂Cl₂, ppm) δ 20.6, 24.9, 30.2, 34.2, 38.3, 40.0, 47.1, 119.3, 120.3, 127.6, 130.5, 133.3, 133.8, 145.7, 155.0, 157.8, 158.2, 164.3, 172.7. FAB-MS m/e 688 (M⁺). UV-VIS (CH₂Cl₂, λₘₐₓ/nm, log ε) 339 (4.57), 399 (3.95), 524 (3.94) 569 (sh, 3.90). Elemental analysis calc. for C₃₆H₃₄N₄O₄Ru.H₂O (%) C, 61.27; H, 5.14; N, 7.94; found C, 61.65; H, 5.18; N, 7.80.

### Example 27

Synthesis of the Ruthenium Complex (VI-3) [Ru(tpy-myrt)(Pydic)]:

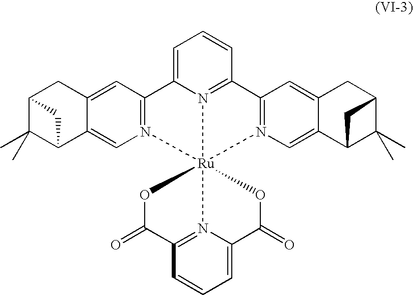

The synthesis is carried out as described under Example 26 using 211 mg of the ligand tpy-myrt (0.50 mmol), 105 mg of Na₂pydic (0.50 mmol) and 152 mg of [Ru(p-cymene)Cl₂]₂ (0.25 mmol). This gave 300 mg of Ru(tpy-myrt)(pydic) (VI-3) (90%) as a violet, crystalline solid.

R_{f}=0.28 (CH₂Cl₂/MeOH 100:5). ¹H NMR (400.1 MHz, CD₂Cl₂): δ 0.62 (s, 6H), 1.14-1.17 (m, 2H), 1.35 (s, 6H), 2.28-2.32 (m, 2H), 2.62-2.64 (m, 4H), 3.24-3.26 (m, 4H), 7.09 (s, 2H), 7.76 (t, J=8.0 Hz, 1H), 8.05 (s, 2H), 8.09 (t, J=7.8 Hz, 1H), 8.25 (d, J=8.0 Hz, 2H), 8.31 (d, J=7.8 Hz, 2H); ¹³C NMR (100.6 MHz, CD₂Cl₂): δ 21.1, 25.5, 31.3, 32.8, 38.9, 39.8, 44.9, 119.6, 121.5, 127.3, 129.2, 133.4, 145.0, 146.5, 146.7, 151.1, 157.6, 157.7, 172.1; FAB-MS (E.I., 70 eV) m/e 688 (M+); UV-VIS (CH₂Cl₂, λₘₐₓ/nm, log ε) 332 (4.58), 392 (4.08), 518 (4.01). Elemental analysis calc. for C₃₆H₃₄N₄O₄Ru.0.5CH₂Cl₂ (%) C, 60.04; H, 4.83; N, 7.67; found C, 59.91; H, 5.08; N, 7.86.

### Example 28

Synthesis of the Ruthenium Complex (VI-4) [Ru(tpy-Me₂-β-Pinene)(Pydic)]:

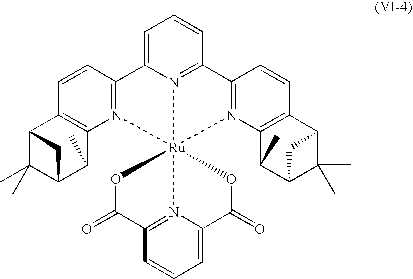

tpy-Me₂-β-pinene (50 mg, 0.11 mmol) and RuCl₃.xH₂O (29 mg, 0.11 mmol) were stirred overnight in n-butanol at 125° C. Pyridine-2,6-dicarboxylic acid (19 mg, 0.11 mmol) and triethylamine (46.6 μl, 0.33 mmol) were then added and the reaction mixture was stirred at 125° C. for another one hour. After removal of the solvent under reduced pressure, the product was purified by chromatography (silica gel using 100:2 to 100:5 CH₂Cl₂:methanol as gradated eluent) to give 41 mg of (VI-4) as a violet, crystalline solid (57%). The analytically pure substance was obtained by recrystallization from CH₂Cl₂/n-hexane.

R_{f}=0.12 (CH₂Cl₂/MeOH 100:5). ¹H NMR (400.1 MHz, CD₂Cl₂, ppm) δ 0.54 (s, 6H), 0.60 (d, J=6.9 Hz, 6H), 1.17 (d, J=10.3 Hz, 2H), 1.32 (s, 6H), 1.73 (m, 2H), 1.94 (ddd, J=13.8, 6.9, 3.6 Hz, 2H), 2.35 (m, 2H), 2.70 (m, 2H), 7.23 (d, J=7.8 Hz, 2H), 7.81 (t, J=8.1 Hz, 1H), 7.92 (d, J=7.8 Hz, 2H), 8.09 (t, J=7.7 Hz, 1H), 8.18 (d, J=8.1 Hz, 2H), 8.31 (d, J=7.7 Hz, 2H). ¹³C NMR (100.6 MHz, CD₂Cl₂, ppm) δ 20.9, 21.1, 25.2, 27.3, 37.9, 40.5, 47.0, 48.1, 119.2, 120.7, 127.2, 131.6, 133.7, 134.1, 144.5, 155.7, 158.6, 159.1, 169.7, 173.2. FAB-MS n/e 716 (M+). UV-VIS (CH₂Cl₂, λₘₐₓ/nm, log ε) 338 (4.54), 396 (3.94), 522 (3.89). HRMS calc. for (C₃₈H₃₈N₄O₄¹⁰²Ru+H⁺) m/e 758.22803 found 758.22870.

The ligands tpy-β-pinene, tpy-myrt and tpy-Me₂-β-pinene 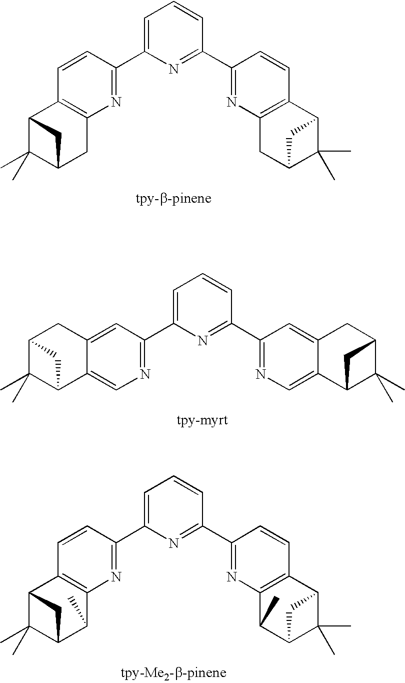
can be prepared, for example, as described by Ziegler, M.; Monney, V.; Stoeckli-Evans, H.; Von Zelewsky, A.; Sasaki, I.; Dupic, G.; Daran, J. C.; Balavoine, G. G. A. Dalton Trans. 1999, 667-675 or Kwong, H.-L.; Lee, W.-S. Tetrahedron: Asymmetry 2000, 11, 2299-2308.

### Example 29

Synthesis of the Ruthenium Complex (VI-5) [Ru(tpy-cam)(Pydic)]:

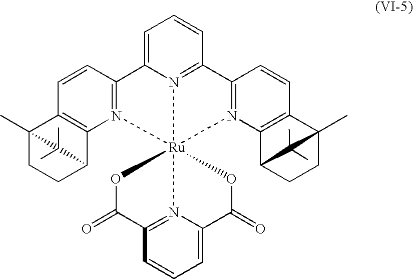

tpy-cam (137 mg, crude product, 0.30 mmol) (for preparation, cf. Example 30) and RuCl₃.xH₂O (80 mg, 0.30 mmol) were stirred overnight in n-butanol at 125° C. Pyridine-2,6-dicarboxylic acid (61 mg, 0.30 mmol) and Et₃N (128 μl, 0.91 mmol) were then added and the reaction mixture was stirred for another one hour. After removal of the solvent under reduced pressure, the product was purified by chromatography (silica gel using 100:0 to 100:5 CH₂Cl₂:methanol as gradated eluent) to give 18 mg of (VI-5) as a violet, crystalline solid (18 mg, 8%). The analytically pure substance was obtained by recrystallization from CH₂Cl₂/n-hexane.

R_{f}=0.18 (CH₂Cl₂/MeOH 100:5). ¹H NMR (400.1 MHz, CD₂Cl₂, ppm) δ 0.35 (s, 6H), 0.73 (s, 6H), 0.83-0.87 (m, 2H), 1.01-1.07 (m, 2H), 1.21 (s, 6H), 1.39 (d, J=3.89 Hz, 2H), 1.71-1.77 (m, 4H), 7.38 (d, J=7.5 Hz, 2H), 7.73 (t, J=7.2 Hz, 1H), 8.07 (d, J=7.7 Hz, 2H), 8.12 (t, J=7.7 Hz, 1H), 8.22 (d, J=7.5 Hz, 2H), 8.34 (d, J=7.7 Hz, 2H). ¹³C NMR (100.6 MHz, CD₂Cl₂, ppm) δ 10.7, 18.1, 19.4, 25.6, 32.3, 52.1, 53.4, 56.3, 119.7, 120.0, 126.5, 127.6, 130.5, 133.1, 148.9, 153.8, 156.3, 157.8, 172.5, 174.3. FAB-MS m/e 717 (M+H⁺). UV-VIS (CH₂Cl₂, λₘₐₓ/nm, log ε) 335 (4.49), 386 (3.90), 519 (3.85). HRMS calc. for (C₃₈H₃₈N₄O₄¹⁰²Ru+H⁺) m/e 717.20148 found 717.20068.

### Example 30

Synthesis of the Ligand tpy-cam: 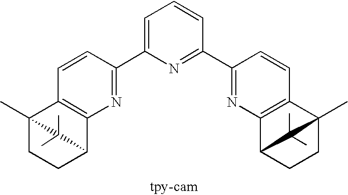
a) Preparation of 2-methylenebornane (cf. Greenwald, R.; Chaykovsky, E. J.; Corey, E. J. J. Org. Chem. 1962, 28, 1128-1129.):

Methyltriphenylphosphonium bromide (17.9 g, 0.05 mmol) dissolved in DMSO (50 ml) and camphor (6 g, 0.04 mmol) dissolved in DMSO (20 ml) were added to a solution of sodium hydride (1.2 g, 0.06 mmol) in DMSO (20 g) at room temperature. This mixture was then stirred at 60° C. for 72 hours. After hydrolysis (50 g of water) and extraction with n-pentane, the organic solvent was removed and the residue was chromatographed (silica gel, n-pentane) to give colourless crystals of 2-methylenebornane (2.12 g, 36%).

Melting point: 65-67° C.; ¹H NMR (400.1 MHz, CD₂Cl₂): δ 0.75 (s, 3H), 0.89 (s, 3H), 0.91 (s, 3H), 1.14-1.27 (m, 2H), 1.60-1.68 (m, 1H), 1.70-1.83 (m, 2H), 1.87-1.94 (m, 1H), 2.35-2.43 (m, 1H), 4.61-4.70 (m, 2H); ¹³C NMR (100.6 MHz, CD₂Cl₂): δ 12.6, 19.0, 19.7, 28.3, 35.5, 37.3, 45.1, 47.5, 51.8, 101.1, 160.1; [α]_{D}=−41.75° (CH₂Cl₂, c=0.92); MS (E.I., 70 eV): m/e 151 ([M+1]⁺, 3), 150 ([M]⁺, 25), 135 (54), 121 (28), 108 (21), 107 (100), 95 (37), 94 (52), 93 (61), 91 (48), 79 (68); HRMS calc. for C₁₁H₁₈ m/e 150.14085 found 150.13664.

b) Preparation of 2-methylene-3-oxobornane (cf. Hartshorn, M. P.; Wallis, A. F. A. J. Chem. Soc. 1964, 5254-5260):

2-Methylenebornane (1.89 g, 12.6 mmol) and selenium dioxide (1.4 g, 12.6 mmol) in carbon tetrachloride (5 ml) were refluxed for 14 hours. The solvent was removed from the reaction mixture and the residue was chromatographed (silica gel, n-hexane) to give light-yellow crystals of 2-methylene-3-oxobornane (873 mg, 42%).

Melting point: 69-75° C.; Rf=0.4 (n-hexane, silica gel); ¹H NMR (400.1 MHz, CD₂Cl₂): δ 0.82 (s, 3H), 0.96 (s, 3H), 1.09 (s, 3H), 1.37-1.46 (m, 2H), 1.86 (dd, J=10.5 Hz, 1H), 1.99 (ddd, J=10.5, 5.2, 2.2 Hz, 1H), 2.20 (d, J=5.2 Hz, 1H), 5.01 (s, 1H), 5.74 (d, J=0.6 Hz, 1H); ¹³C NMR (100.6 MHz, CD₂Cl₂): δ 11.9, 17.3, 20.4, 22.7, 34.5, 45.7, 51.6, 59.3, 110.4, 154.7, 255.7; [α]_{D}=−127.6° (CH₂Cl₂, c=0.64); MS (E.I., 70 eV): m/e 165 ([M+1]⁺, 9), 164 ([M]⁺, 63), 149 (56), 136 (21), 122 (28), 121 (100), 107 (33), 96 (65), 95 (48), 93 (66), 91 (34), 79 (38), 77 (32), 69 (35), 67 (82), 55 (32), 41 (93), 39 (49), 27 (40). HRMS calc. for (C₁₁H₁₆O) m/e 164.12012 found 164.12049.

c) Preparation of tpy-cam:

2-Methylene-3-oxobornane (773 mg, 4.71 mmol), ammonium acetate (726 mg, 9.42 mmol) and 2,6-bis(pyridinoacetyl)pyridine diiodide (1.347 g, 2.35 mmol) (prepared as described by Ziegler, M.; Monney, V.; Stoeckli-Evans, H.; Von Zelewsky, A.; Sasaki, I.; Dupic, G.; Daran, J. C.; Balavoine, G. G. A. Dalton Trans. 1999, 667-675) were suspended in acetic acid (10 ml) and stirred at 120° C. in a pressure tube for 14 hours. After neutralization of the reaction mixture by means of 16% strength sodium carbonate solution and extraction with chloroform, the organic solvent was removed to give the ligand tpy-cam (137 mg, crude product).

### Example 31

Asymmetric Epoxidation of 1-Phenyl-2-Methylpropene Using the Complex (VI-3) as Catalyst

The ruthenium complex (VI-3) (0.0025 mmol) was stirred in tert-amyl alcohol (9 ml) at room temperature and 1-phenyl-2-methylpropene (0.5 mmol) was added. A solution of 30% strength hydrogen peroxide (1.5 mmol) in t-amyl alcohol (0.83 ml) was metered into this mixture over a period of 12 hours. The reaction was then quenched by addition of water (10 ml) and Na₂SO₃ (0.5 g) and the mixture was extracted with ethyl acetate (20 ml). After the organic phase had been dried, the epoxide yield was determined by means of gas chromatography. Yield: 96% of theory; enantiomeric excess=+54% ((R)-(+)-1-phenyl-2-methylpropene oxide is the enantiomer present in excess).

The enantiomeric excess was determined by means of HPLC on a chiral column material and reported as the ee (S) or (R) value defined below.

The ee value is obtained via the following equations:

$$ee ( S ) = \frac{m ( S ) - m ( R )}{m ( S + R )} ⨯ 100 \%$$

$$ee ( R ) = \frac{m ( R ) - m ( S )}{m ( S + R )} ⨯ 100 \%$$

where ee (S) or ee (R) is the optical purity of the S or R enantiomer, m(S) is the molar amount of the enantiomer S and m(R) is the molar amount of the enantiomer R. (Examples: for a racemate: R═S=>ee=0; for the pure (S) form: ee (S)=100%; for a ratio of S:R=9:1, ee (S)=80%).

1. Process for preparing compounds of the formula (I), 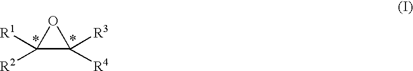
where
R¹, R², R³ and R⁴ are each, independently of one another, hydrogen, alkyl, aryl, arylalkyl, haloalkyl or a radical of one of the formulae (IIa) to (IIf)
A-B-D-E (IIa)
A-E (IIb)
A-SO₂-E (IIc)
A-B-SO₂R⁶ (IId)
A-SO₃W (IIe)
A-COW (IIf)
where, in the formulae (IIa) to (IIf)
A is absent or is an alkylene or haloalkylene radical and
B is absent or is oxygen or NR⁵, where
R⁵ is hydrogen, arylalkyl or aryl, and
D is a carbonyl group and
E is R⁶, OR⁶, NHR⁷ or N(R⁷)₂,
where
R⁶ is alkyl, arylalkyl or aryl and
the radicals R⁷ are each, independently of one another, alkyl, arylalkyl or aryl or the moiety N(R⁷)₂ is a cyclic amino radical having from 4 to 12 carbon atoms, and
W is OH, NH₂, or OM, where M is an alkali metal ion, half an equivalent of an alkaline earth metal ion, an ammonium ion or an organic ammonium ion,
or two of the radicals R¹, R², R³ and R⁴ are together part of a 3- to 7-membered ring having a total of from 3 to 16 carbon atoms,
wherein compounds of the formula (III), 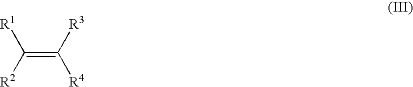
where R¹, R², R³ and R⁴ are, in each case independently of one another, as defined above,
are reacted with hydrogen peroxide,
with the reaction being carried out in the presence of a ruthenium complex which bears as ligands both compounds of the formula (IV) 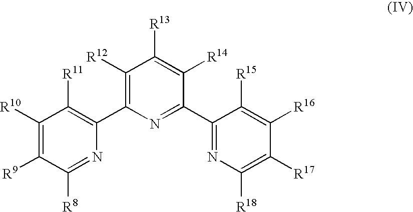
where R⁸, R⁹, R¹⁰, R¹¹, R¹², R¹³, R¹⁴, R¹⁵, R¹⁶, R¹⁷ and R¹⁸ are each, independently of one another, hydrogen, halogen, hydroxy, hydroxycarbonyl, alkoxycarbonyl, alkoxy, alkyl, arylalkyl or aryl, or
two of the radicals R⁸, R⁹, R¹⁰ and R¹¹ or two of the radicals R¹⁵, R¹⁶, R¹⁷ and R¹⁸ are together part of a 3- to 7-membered monocycle having a total of from 3 to 16 carbon atoms or are together part of a bicycle having a total of from 3 to 16 carbon atoms,
and also compounds of the formula (V) 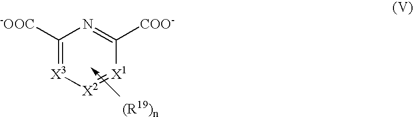
where
X¹, X² and X³ are each, independently of one another, N, CH or CR¹⁹ and
R¹⁹ is hydrogen, halogen, hydroxy, hydroxycarbonyl, alkoxycarbonyl, alkoxy, alkoxyalkyl, arylalkyl or aryl and
n is 0, 1, 2 or 3, preferably 0 or 1 and particularly preferably 0.

2. Process according to claim 1, wherein the formula (I), R¹, R², R³ and R⁴ are each preferably, independently of one another, hydrogen, substituted or unsubstituted C₁-C₈-alkyl, substituted or unsubstituted C₅-C₁₄-aryl, substituted or unsubstituted C₆-C₁₅-arylalkyl or C₁-C₈-haloalkyl or two of the radicals R¹, R², R³ and R⁴ are together part of a 3- to 7-membered ring having a total of from 3 to 16 carbon atoms.

3. Process according to claim 1 wherein the formula (I), at least one radical R¹, R², R³ and R⁴ is substituted or unsubstituted C₅-C₁₄-aryl or two of the radicals R¹, R², R³ and R⁴ are together part of a 3- to 7-membered ring having a total of from 3 to 16 carbon atoms.

4. Process according claim 1 wherein the formula (IV), R⁸, R⁹, R¹¹, R¹², R¹⁴, R¹⁵, R¹⁷ and R¹⁸ are each hydrogen and at the same time R¹⁰, R¹³ and R¹⁶ are each tert-butyl, R⁸, R⁹, R¹⁰, R¹¹, R¹², R¹³, R¹⁴, R¹⁵, R¹⁶, R¹⁷ and R¹⁸ are each hydrogen or two of the radicals R⁸, R⁹, R¹⁰ and R¹¹ and two of the radicals R¹⁵, R¹⁶, R¹⁷ and R¹⁸ are together part of a 3- to 7-membered monocycle having a total of from 3 to 16 carbon atoms or are together part of a bicycle having a total of from 3 to 16 carbon atoms and the remaining radicals R⁸, R⁹, R¹⁰, R¹¹, R¹², R¹³, R¹⁴, R¹⁵, R¹⁶, R¹⁷ and R¹⁸ are each hydrogen.

5. Process according to claim 1 wherein the formula (IV), R⁸, R⁹, R¹⁰, R¹¹, R¹², R¹³, R¹⁴, R¹⁵, R¹⁶, R¹⁷ and R¹⁸ are each hydrogen.

6. Process according to claim 1 wherein the formula (V), at least two, preferably three, of the radicals X¹, X², X³ are CH or CR¹⁹.

7. Process according to claim 1 wherein ruthenium complexes used are complexes of the formula (VI)
[Ru(IV)(V)] (VI)
where (IV) represents a compound of the formula (IV) and (V) represents a compound of the formula (V), or complexes which are generated in situ in the reaction mixture from a suitable ruthenium precursor and the two ligands of the formulae (IV) and (V).

8. Process according to claim 1 wherein it is carried out in the presence of secondary or tertiary alcohols as solvents.

9. Compounds of the formulae (VI-2) to (VI-5) [chemical structure]
